(19) United States
(12) Patent Application Publication
(10) Pub. No.: US 2002/0044886 A1
(43) Pub. Date: Apr. 18, 2002

(54) Vaporization system having blower positioned in reduced temperature zone

(76) Inventor: William Larco, Houston, TX (US)

Correspondence Address: CONLEY ROSE & TAYON, P.C., P. O. BOX 3267, HOUSTON, TX, 77253-3267, US

(21) Appl. No.: 09/978,918

(22) Filed: Oct. 16, 2001

Related U.S. Application Data
(60) Provisional application No. 60/240,725, filed on Oct. 16, 2000.

(51) Int. Cl. A61L 9/00; A62B 7/08

(52) U.S. Cl. 422/28; 422/4; 422/5; 422/29; 422/33; 422/123; 422/124

(57) ABSTRACT

A pollution control system having a blower located in a reduced temperature zone is disclosed. In one embodiment, the system comprises: an injection grid, a vaporizer, and a blower. The injection grid injects ammonia vapor into a flue gas stream. The ammonia vapor is created as the vaporizer vaporizes aqueous ammonia into a carrier medium. Advantageously, the vaporization of the aqueous ammonia reduces the temperature of the carrier medium. A blower moves the carrier medium flow through the vaporizer and into the injection grid. The blower is located downstream of the vaporizer and upstream of the injection grid. This location permits the blower to operate in an environment with a substantially reduced temperature relative to the prior art. This advantageously increases the reliability of the blower and reduces installation and maintenance costs.

### CROSS-REFERENCE TO RELATED APPLICATIONS

This application claims priority to provisional application Ser. No. 60/240,725, filed Oct. 16, 2000, which is hereby incorporated herein by reference.

### BACKGROUND OF THE INVENTION

The present invention relates in general to systems for vaporizing fluids. In particular, the present invention relates to a system suitable for vaporization of aqueous ammonia to reduce nitrogen oxide (NOₓ) levels in flue gases. Even more particularly, the present invention relates to a system configuration that reduces operating and installation costs and improves reliability by locating any blowers in system zones where the temperature is reduced.

A flue gas stream is formed during the combustion of fuels such as coal, oil, natural gas, petroleum coke, etc., which are burned by electric power generating plants and many other industrial processes. The combustion process creates NO₂ and NO₃, commonly called NOₓ. Most industries use a selective catalytic reduction (SCR) system to remove NOₓ from the flue gas. The NOₓ removal process involves introducing an ammonia reagent into the flue gas. The flue gas and the ammonia reagent travel through a catalytic converter that facilitates the breakdown of NOₓ into nitrogen (N₂), oxygen (O₂), and water. There are several known methods used to remove NOₓ from the flue gas in SCR reactors.

A first known method uses anhydrous ammonia in order to reduce NOₓ levels. The anhydrous ammonia is evaporated with either an electric source or with steam coils. The vaporized ammonia is then diluted with air in order to provide an adequate mass necessary to distribute the ammonia reagent evenly over a large ductwork cross-section. In this method, the diluted ammonia and air mixture is delivered to a grid of injection pipes located in the flue gas ductwork and upstream of a SCR catalyst bed. The injection pipes span the width of the flue gas duct and are closed at one end. The ammonia and air mixture is injected into the flue gas through nozzles or orifices that are sufficiently spaced along the injection pipes in order to provide an even distribution and thorough mixing of the ammonia with the flue gas. Major disadvantages associated with using this method include the safety concerns and precautions pertaining to the handling and storage of the anhydrous ammonia. Especially in highly populated areas, local government regulations often require that aqueous ammonia be used instead of anhydrous ammonia.

A second method for reducing NOₓ levels is to use an aqueous ammonia with an external heat source in order to evaporate the aqueous ammonia. The aqueous ammonia used is typically purchased in industrial grade form and is approximately 16-30% by weight ammonia and 84-70% by weight water. A dedicated heater, usually an electric-type heater, is used to heat dilution air to a level which is adequate enough to vaporize the ammonia and water mixture. A vaporization chamber or static mixer is the medium in which the phase change occurs. Usually, atomization air is required to assist in the break-up of the aqueous ammonia in order for fine droplets of the aqueous ammonia to enter the vaporization chamber. After vaporization, the ammonia and water air mixture exits the vaporization chamber and is delivered to an injection grid for injection into the flue gas as described above.

A major disadvantage associated with this method is that there is a parasitic power demand caused by the dilution air heater. A typical installation can have heater power demands ranging in the hundreds of KW range. Furthermore, there is great cost associated with this method due to the capital cost of the air heater and associated controls and hardware. Additionally, there are several maintenance problems associated with this method, particularly, burned-out heating elements which lead to costly maintenance down time.

A third method is to vaporize aqueous ammonia using the heat energy from the flue gas. This method comprises taking a hot slip stream of the flue gas from the ductwork, upstream of the SCR reactor, and in turn sending it through a vaporization chamber by means of a high temperature fan or blower. As described in the second known method above, the aqueous ammonia is injected into the vaporization chamber with atomization air in order to facilitate the phase change. As previously described, the ammonia-water-flue gas mixture exits the vaporization chamber and is delivered to an injection grid. The major disadvantages associated with this method include the cost and low reliability of the high temperature fans or blowers.

A fourth known method for reducing NOₓ in a flue gas is to spray aqueous ammonia directly into the flue gas upstream of the SCR catalyst bed. In this method, the aqueous solution is sprayed into the flue gas upstream of the catalyst bed in a manner similar to the way reagent is introduced into a selective non-catalytic reduction process (SNCR) in which a liquid ammonia derivative is sprayed in boiler high temperature regions in order to accomplish NOₓ reduction. The energy from the flue gas is used to accomplish the phase change.

A major problem associated with this method is that great residence time is required in order to vaporize the water and ammonia. Additionally, there may be insufficient distance upstream of the catalyst bed for placing the injection pipes. This is further complicated by the requirement of protecting the SCR catalyst from liquid water in order to avoid contamination. Through this method, the need to provide carrier mass is not met which means that the number of total nozzles in the cross-section of the flue gas is limited. Thus, this method limits the capability to have a uniform injection distribution.

### SUMMARY OF THE INVENTION

The problems outlined above are at least in part solved by a pollution control system having a blower located in a reduced temperature zone. In one embodiment, the system comprises: an injection grid, a vaporizer, and a blower. The injection grid injects ammonia vapor into a flue gas stream. The ammonia vapor is created as the vaporizer vaporizes aqueous ammonia into a carrier medium. Advantageously, the vaporization of the aqueous ammonia reduces the temperature of the carrier medium. A blower moves the carrier medium flow through the vaporizer and into the injection grid. The blower is located downstream of the vaporizer and upstream of the injection grid. This location permits the blower to operate in an environment with a substantially reduced temperature relative to the prior art. This advantageously increases the reliability of the blower and reduces installation and maintenance costs.

### BRIEF DESCRIPTION OF THE DRAWINGS

A better understanding of the present invention can be obtained when the following detailed description of the preferred embodiment is considered in conjunction with the following drawings, in which:

FIG. 1 shows an exemplary pollution control system in the context of a commercial furnace; and

FIG. 2 shows a preferred vaporization system embodiment.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENT

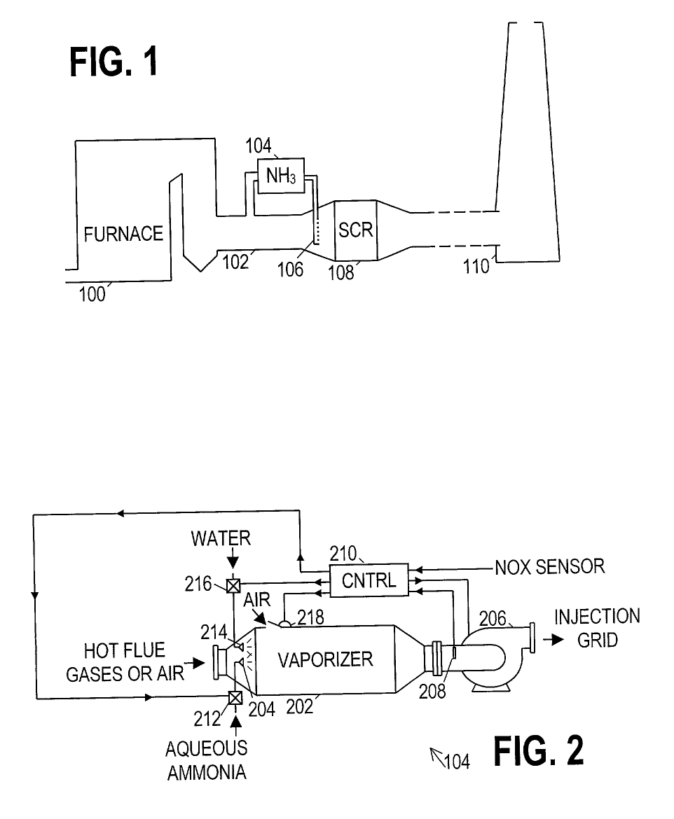

Turning now to the figures, FIG. 1 shows a furnace 100 in which the burning of combustible fuels occurs. Gas byproducts of the combustion pass into a flue duct 102, past an ammonia vapor injection grid 106, through a SCR region 108, and eventually out a stack 110 into the atmosphere. Injection grid 106 receives a mixture of gases including ammonia vapor from a vaporization system 104, described in more detail below. The diluted ammonia vapor mixes with the flue gases and as the mixture passes through the SCR region 108, catalysts cause the ammonia to react with the NOₓ in the flue gas to produce harmless byproducts of nitrogen and water vapor.

FIG. 2 shows a preferred embodiment of vaporization system 104. The vaporization system 104 may take hot flue gases from duct 102 to use as a carrier medium for the ammonia vapor. However, other carrier mediums may alternatively be used including, e.g., heated air. The temperature of the carrier medium is generally expected to exceed 700° F., and indeed, may be expected to reach 800 or 900° F.

As the carrier medium passes into a vaporizer 202, aqueous ammonia is directly injected via an array of spray nozzles 204. Heat from the carrier medium is absorbed by the droplets of aqueous ammonia spray, causing the droplets to vaporize and at the same time reducing the carrier medium temperature. The temperature of the gas mixture leaving the vaporizer 202 is expected to fall below 300° F., and is preferably limited to less than 350° F.

Motion of the carrier medium into and through the vaporizer 202 is induced by a blower 206 located downstream from the vaporizer and upstream from the injection grid 106. It is noted that this placement of the blower 206 advantageously reduces the performance requirements of the blower. If the blower were placed upstream of the vaporizer, it would have to cope with much higher operating temperatures and flow.

The reduction in operating temperature is expected to significantly extend the operating life of the blower while simultaneously reducing the cost. As an additional advantage, the reduction in temperature will reduce the specific volume of the gas mixture downstream of the vaporizer. The mass transfer that would be provided by a blower upstream of the vaporizer can therefore be provided by a smaller blower downstream from the vaporizer.

A controller 210 is preferably provided to ensure optimal NOₓ removal with minimal wastage of aqueous ammonia. It preferably includes an input from a NOₓ sensor downstream from the SCR 108. The controller 210 regulates the flow of injected aqueous ammonia, preferably via a valve 212, to provide the optimum amount of diluted ammonia through the injection grid.

Since blower 206 is rated for lower temperature operation, the preferred embodiment includes means to protect the blower 206 by preventing the temperature of the carrier medium exiting the vaporizer 202 from exceeding the rated temperature, e.g. 350° F. To this end, the controller 210 is provided with a temperature sensor 208 near the inlet of blower 206. If the temperature sensor indicates that the operating temperature of the blower is approaching or exceeding a threshold temperature, the controller 210 can actuate one or more systems to reduce the operating temperature.

A first way that may be used to reduce the operating temperature of the blower is a second injector 214 that injects water into the vaporizer. If the temperature is rising because the aqueous ammonia flow has been reduced, the controller may compensate by opening valve 216 to inject more water into the vaporizer. The increased mass of water being vaporized by the carrier medium will cool the mixture entering the blower 206.

A second way that may be used to reduce the operating temperature of the blower 206 is a damper 218. Opening the damper allows ambient air to enter the vaporizer 202 and dilute the carrier medium. This will also cool the mixture entering the blower 206.

Yet another way to reduce the operating temperature of the blower 206 is to alter the blower speed. Slowing or stopping the blower 206 will slow the movement of the carrier medium, thereby allowing more complete vaporization of injected fluids, and/or allowing heat loss through the walls of the vaporizer 202 and upstream ducts of vaporization system 104.

Through any one of these or other methods or combinations thereof, controller 210 may limit the operating temperature of the blower 206. It is expected, however, that the blower 206 will be primarily protected by the atemporization effect of the vaporization of injected aqueous ammonia. This is expected to reduce the carrier medium temperature by at least 200° F. and more preferably it will reduce the carrier medium temperature by 400° F. or more.

Numerous variations and modifications will become apparent to those skilled in the art once the above disclosure is fully appreciated. It is intended that the following claims be interpreted to embrace all such variations and modifications.

What is claimed is:

1. A pollution control system that comprises:
an injection grid configured to inject ammonia vapor into a flue gas stream;
a vaporizer configured to vaporize aqueous ammonia into a carrier medium, thereby reducing the temperature of the carrier medium; and
a blower coupled between the vaporizer and the injection grid, wherein the blower is configured to induce motion of the carrier medium through the vaporizer, the blower, and the injection grid.

2. The pollution control system of claim 1, wherein said injection grid comprises a plurality of apertures configured to deliver a vaporized mixture of aqueous ammonia and flue gas.

3. The pollution control system of claim 1, wherein said injection grid comprises a plurality of apertures configured to deliver a vaporized mixture of aqueous ammonia and air.

4. The pollution control system of claim 1, wherein said carrier medium comprises flue gas.

5. The pollution control system of claim 1, wherein said carrier medium comprises heated air.

6. The pollution control system of claim 1 wherein said vaporizer comprises an array of apertures configured to inject aqueous ammonia directly into said carrier medium.

7. The pollution control system of claim 1 wherein the heat from the carrier medium is sufficient to vaporize said aqueous ammonia.

8. The pollution control system of claim 1, wherein the vaporizer reduces the temperature of the carrier medium by at least 200° F.

9. A method for controlling pollution comprising:
configuring an injection grid to inject ammonia vapor into a flue gas stream;
configuring a vaporizer to vaporize aqueous ammonia into a carrier medium, thereby reducing the temperature of the carrier medium; and
coupling a blower between said vaporizer and injection grid.

10. The method of claim 9 wherein said blower is configured to induce motion of the carrier medium through the vaporizer, the blower, and the injection grid.

11. The method of claim 9 wherein said blower draws flue gases from the flue duct into the vaporizer.

12. The method of claim 9 wherein said carrier medium comprises flue gases existing in said flue duct.

13. The method of claim 9 wherein said carrier medium comprises heated air.

14. A method of reducing NOₓ concentrations in a flue gas stream, the method comprising:
injecting aqueous ammonia fluid into a carrier medium flow in a vaporizer;
drawing the carrier medium flow with ammonia vapor through a blower; and
dispersing the carrier medium flow with ammonia vapor in the flue gas stream.

15. The method of claim 14, further comprising:
exposing the flue gas stream with dispersed ammonia vapor to a SCR catalyst.

16. The method of claim 15, wherein the carrier medium is flue gas.

17. The method of claim 15, wherein the carrier medium is heated air.

18. The method of claim 15, wherein the vaporizer reduces the temperature of the carrier medium by at least 200° F.

19. The method of claim 15, wherein the vaporizer reduces the temperature of the carrier medium by at least 400° F.
